(19) United States
(12) Patent Application Publication
(10) Pub. No.: US 2012/0156153 A1
(43) Pub. Date: Jun. 21, 2012

(54) Naturally Derived, Polymeric Hair Fixative Systems With Pullulan, And Mascara Compositions Comprising The Same

(76) Inventors: Stanley R. Fogg, Shoreview, MN (US); Divyesh Patel, Forest Lake, MN (US); Cindy L. Orr, Blaine, MN (US)

(21) Appl. No.: 13/166,917

(22) Filed: Jun. 23, 2011

Related U.S. Application Data
(60) Provisional application No. 61/360,154, filed on Jun. 30, 2010.

(51) Int. Cl. A61K 8/92; A61Q 1/10

(52) U.S. Cl. 424/70.7

(57) ABSTRACT

A naturally-derived, polymeric hair fixative system that is suitable for use in commercially acceptable mascara compositions. The hair fixative system comprises a natural, polymeric film forming system that comprises pullulan and candelilla wax resin, and a naturally derived plasticizer system that comprises one or more rosins and or rosin derivatives, and a sebacic acid/castor oil copolymer. The hair fixative system is free of synthetic film formers, heavy metals, crosslinking ingredients, catalysts, and complexes that may be harmful to the user and the environment. This hair fixative system enables a natural approach to mascara formulation, without the drawbacks of shellac-based systems, systems based solely on pullulan, or synthetic systems.

### CROSS-REFERENCE TO RELATED APPLICATIONS

This application claims priority from U.S. Provisional Application No. 61/360,154, filed Jun. 30, 2010.

### FIELD OF THE INVENTION

The present invention relates to mascara formulations that are based on natural ingredients or naturally derived ingredients, and that provide a continuous film, with little or no flaking and smearing. More specifically, the present invention relates to naturally derived resins and film formers used as eyelash fixatives, which serve as a base for natural mascaras or mascaras with significantly less synthetic material.

### BACKGROUND

In cosmetics, hair fixatives are used to hold hair in a shape for an extended period of time. Better fixatives are strong enough to withstand incidental movement of the hair, and resilient enough to return to a pre-determined shape. At the same time, a good hair fixative will break down with combing or brushing, and have good removability with washing, but not be too susceptible to ambient humidity. Better hair fixatives also provide good hold, with a less sticky feel. Other properties of better hair fixatives include gloss, non-flakiness, compatibility with the composition vehicle, and water solubility.

Film forming agents, polymers in particular, have a number of properties that make them very useful in hair fixative systems. Film forming polymers form a more or less resilient, continuous coating on a body surface, such as skin or hair. They can be applied wet, allowing some time to style the hair, but upon drying they form a continuous film that provides a measure of hold to the hair.

In general, mascara compositions that are based on linear film forming polymers are less susceptible to flaking than mascara compositions based on non-linear film forming polymers. Likewise, mascara compositions that are based on film forming polymers that have relatively less branching are less susceptible to flaking than mascara compositions based film forming polymers that have relatively more branching.

There are natural polymer film formers and synthetic polymer film formers. For various environmental and safety reasons that are generally acknowledged, natural materials are often preferred to synthetic materials. For example, synthetic materials may be harmful to the environment when products containing them are discarded; they often require more energy to produce; and a user may have an adverse reaction to a synthetic material. However, for several reasons synthetic polymer film formers have become ubiquitous. First, there are relatively few naturally derived film forming polymers that are also water soluble, non-animal derived, and suitable for cosmetic use. Also, there are relatively few natural or naturally derived film forming polymers that are linear and that have little or no branching. Thus, from a mascara performance perspective, synthetic film formers are preferred because the linearity and degree of branching can be controlled.

One example of a natural film forming polymer that has been used in mascara, is shellac. Shellac is a natural, albeit animal based polymer, derived from the secretion of the insect Laccifer lacca. Shellac is soluble in alkaline solutions and various organic solvents, but insoluble in water. However, since the trend in commercial hair fixatives, for some time now, is away from volatile solvents in the carrier system, when shellac is used, it must first be solubilized for use in water-based systems. For example, solubility in water may be improved by forming a salt of shellac. But this approach does not always provide satisfactory results, and eventually, formulators of commercial mascara compositions, came to rely on synthetic polymeric film formers having greater water solubility. Also, shellac is animal-derived, which is not acceptable to all consumers.

Another natural film forming polymer that is sometimes used in mascara is pullulan. Pullulan is produced from starch by the fungus Aureobasidium pullulans. It is a water soluble material known for its ability to form transparent films with high gloss and good adhesive strength. Pullulan has been used in the food and personal care industries. One of the first reported uses of pullulan in cosmetic products may have been JP 48-116652, filed Oct. 16, 1973. The corresponding US patent (U.S. Pat. No. 3,972,997, herein incorporated by reference), discloses:
“A cosmetic can be improved and enhanced in its effect when incorporated with pullulan, which is a linear high polymer having repetition units, bonded through α-1,6-linkages, of maltotriose, a trimer of glucose, and is represented by the formula,

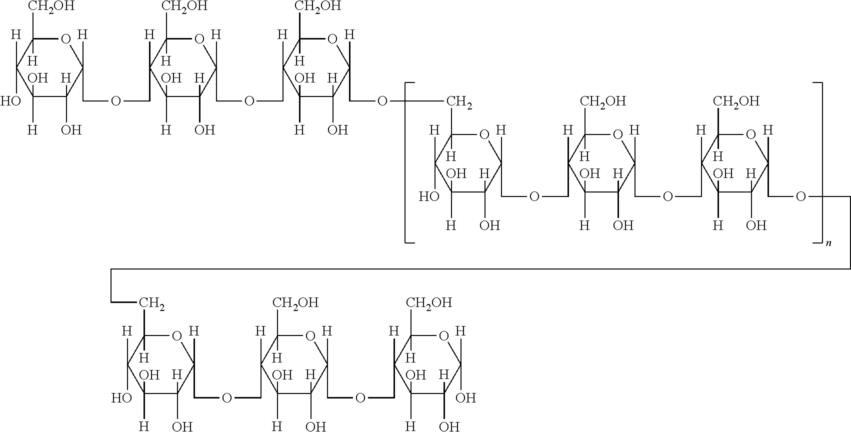

wherein n represents the polymerization degree and is an integer of 10 to 10,000.”
   It is further disclosed:
“ . . . pullulan may be applied to any cosmetics, but is preferably used as an ingredient of . . . cosmetic around the eyes, specific hair dressings (set lotions and hair lacquers) . . . ” That is, the pullulan can be used . . . as an ingredient of cosmetics around the eyes, e.g. eye liners, taking advantage of its non-toxicity, non-irritability and film-forming ability . . . as an ingredient of set lotions and hair lacquers, taking advantage of its tackiness, ability of forming tough film, hair setting ability, and high water solubility necessary for removal after use”.

In U.S. Pat. No. 3,972,997, mascara is not specifically mentioned. Nevertheless, mascara products comprising pullulan are known. However, to the best of the applicant's knowledge, formulating a commercially acceptable mascara composition with a pullulan film former, and no synthetic film formers, has not been achieved. In our experience, when pullulan is the sole film former in the mascara, the quality of the mascara is not commercially acceptable. Even though pullulan is linear and has relatively little branching compared to other natural film forming polymers, mascara compositions with pullulan, and no other film formers, tend to smudge. Also, for some time after application, mascara compositions with pullulan as the sole film former tend to be hard and brittle, which leads to flaking. Thus, when pullulan has been used in commercial mascara, synthetic film formers are also present.

In fact, eyelash products have come to rely on the use of synthetic polymers. Many commercial mascara formulas use petroleum-based synthetic polymers or copolymers. For example, it is common to use materials based on combinations of polyvinyl pyrrolidone (PVP), and vinyl acetate (VA), or other co-monomers. The addition of vinyl acetate to PVP makes the resulting film less susceptible to atmospheric moisture. However, the PVP/VA film is difficult to remove from hair, such as the eye lashes, and has a sticky feel. To address the removability problem, formulators mix PVP/VA with one or more resins to improve removability with washing.

Thus, there remains a need for a commercially acceptable mascara composition that has no synthetic film formers. Surprisingly, it has been discovered that combinations of pullulan and a resin from Candelilla wax make an effective natural film forming system, and when balanced with a naturally derived plasticizer system comprising a resin from rosin, and a sebacic acid/castor oil copolymer, the result is an excellent, naturally derived hair fixative system, suitable for commercially acceptable mascara products.

There is an established vocabulary for discussing the performance characteristics of mascara. Each of these characteristics can be evaluated and assigned a number on a random scale, from 0 to 10, say, for purposes of comparison during formulation. “Length” is the dimension of the lash from the free tip to its point of insertion in the skin. Increasing length is frequently a goal of mascara application. “Volume” (or fullness) depends on the volume of the lashes and the space the between them, where “sparse” (or less full) means there are relatively fewer lashes and relatively larger separation between the lashes and “dense” (or more full) means the lashes are tightly packed with little measurable space between adjacent lashes. “Curl” is the degree to which a mascara causes upward arching of the lashes relative to the untreated lashes. Curl is often desirable. “Separation” is the non-aggregation of lashes so that each individual lash is well defined. Good separation is one of the desired effects of mascara application. “Thickness” is the diameter of an individual lash, which may be altered in appearance by the application of mascara. Increasing thickness is usually a goal of mascara application. “Spiking” is the tendency for the tips of individual lashes to fuse, creating a triangular shaped cluster, usually undesirable. “Clumping”, as a result of mascara application, is the aggregation of several lashes into a thick, rough-edged shaft. Clumping reduces individual lash definition and is generally not desirable. “Flaking” refers to pieces of dried mascara coming off the lashes after defined hours of wear. The better quality mascaras do not flake. “Smudging” is the propensity for mascara to smear after defined hours of wear, when contacting the skin or other surface. Smudging is facilitated by the mascara mixing with moisture and/or oil from the skin or environment and becoming softened. “Wear” is the visual impact of a mascara on the lashes after defined hours as compared to immediately after application, and is directly related to the amount of mascara that remains on the lashes. “Overall look” is one overall score that factors in all the above definitions. It is a subjective judgment comparing treated and untreated lashes or comparing the aesthetic appeal of one mascara to another. The ideal mascara will possess all of the desirable properties while avoiding the undesirable. In reality, a mascara developer may improve one or more characteristics only at the expense of others. For example, to increase the volumizing ability of a particular mascara, a developer may add more solids (i.e. wax) to the composition. However, a disadvantage of doing this is that it tends to increase clumping of the composition, and decrease the user's ability to separate the lashes. Thus, an improvement in less than all of the performance characteristics, sometimes even just one, may represent a substantial improvement over the prior art.

### OBJECTS OF THE INVENTION

A main object of at least some embodiments of the invention is to provide a naturally derived, polymeric hair fixative system that is suitable for use in commercially acceptable mascara compositions.

Another object of at least some embodiments of the invention is to provide a commercially acceptable mascara composition that has an all natural, polymeric film forming system comprising pullulan.

Another object of at least some embodiments of the invention is to provide a mascara composition that has no synthetic film formers, and that is commercially acceptable, especially from the point of view of flaking and smudging.

Another object of at least some embodiments of the invention is to provide a hair fixative system having no synthetic film formers, no heavy metals, no crosslinking ingredients, no catalysts, and no complexes that may be harmful to the user and the environment.

Another object of at least some embodiments of the invention is to provide a commercially acceptable mascara composition comprising a film forming system of pullulan and a resin from Candelilla wax, in combination with a plasticizer system comprising a resin from rosin, and a sebacic acid/castor oil copolymer.

### SUMMARY

The present invention includes eyelash compositions that have a naturally derived, polymeric hair fixative system comprising a film forming system of pullulan and a resin from Candelilla wax, in combination with a plasticizer system comprising a resin from rosin, and a sebacic acid/castor oil copolymer. This combination of materials enables a more natural approach to mascara formulation, without the drawbacks of shellac-based systems, systems based solely on pullulan, or synthetic systems.

### DETAILED DESCRIPTION

### Definitions

Throughout the specification, “comprise” means that an element or group of elements is not automatically limited to those elements specifically recited, and may or may not include additional elements.

Throughout the specification a “natural” material is one which has been harvested, mined or collected, and which subsequently may have been processed, without chemical reaction, to yield a chemical or chemicals that are identifiable in the original source material. The phrase “without chemical reaction” permits washing, decolorizing, distilling, grinding, milling, separating and/or concentrating the material by physical means. By “naturally derived”, we mean materials wherein the majority of the molecule (by weight) is derived from natural materials.

“Plant derived” means that a material or substance is harvested from vegetation (i.e. flowers, plants, trees, herbs, bushes, grasses, vines, ferns, mosses and green algae). According to this definition, a chemically identical material or substance assembled by human agent or derived from an animal, is not “plant derived”.

“Commercially acceptable” means that a mascara composition performs about as well or better, at least in some aspects, than a commercial mascara product sold on the market today. “Commercially acceptable” means especially that the mascara builds volume and does not flake, about as well or better, than a commercial mascara product sold on the market today.

### Pullulan

A first necessary component of the invention is naturally occurring pullulan or naturally occurring derivative of pullulan. Pullulan is a polysaccharide having a primary structure based on maltotriose. Maltotriose (C₁₈H₃₂O₁₆ molecular weight=504.4 g/mol) has three glucose units connected by α-1,4 glycosidic bonds. In pullulan, consecutive maltotriose units are connected to each other by α-1,6 glycosidic bonds. A representation of the primary structure was shown above. The following secondary structure, based on maltotetraose units, has also been reported:

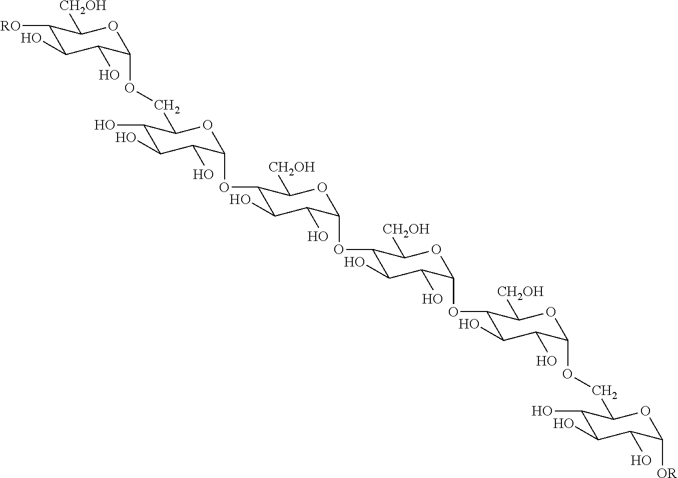

Pullulan is relatively straight, whereas other naturally occurring polysaccharides contain more branched structures and more side chains than pullulan (for example xantham gum or dextran). Pullulan is a binder and film former, in particular, it can be formed into clear, tough, resilient films with good adhesion. Pullulan is water soluble, and therefore, may preferably be included in an aqueous phase of the mascara composition. Pullulan resists gelling in solution, unlike some other naturally occurring polysaccharides (i.e. curdlan). Pullulan having a polymerization degree of 10-10,000 (or molecular weight=about 5,000 to about 50,000) may be useful in the present invention. A useful naturally occurring derivative of pullulan is myristoyl pullulan. The concentration of pullulan in the present invention may be adjusted according to the needs of the specific formulation, but will generally be from 0.1% to 15% by weight of the composition, more preferably 1% to 10%, more preferably still 2% to 5% by weight of the final composition.

As noted above, mascara compositions that are based on linear film forming polymers, with less branching, are, in general, less susceptible to flaking than mascara compositions based on non-linear film forming polymers, with more branching. Nevertheless, even though pullulan is linear and has relatively little branching compared to other natural film forming polymers, we have observed that mascara compositions with pullulan and no other film formers, tend to flake and smudge. At the outset, therefore, it was not clear that an all natural film forming system based on pullulan, could be achieved, since pullulan, which is relatively linear and non-branched, could not do it alone.

### Candelilla Wax Resin

A second component of the invention is a resin from Candelilla wax. Candelilla resin is natural, pliable and provides good adhesion to the eyelashes. Its use in mascara tends to increase volume and decrease smudging and flaking. Candelilla wax is derived from the leaves of the Candelilla shrub (Euphorbia cerifera and Euphorbia antisyphilitica). The present invention makes use of the resins found in the wax. The wax itself is not a required component of the invention. About 12-14% of candelilla wax is resin, reportedly comprised of triterpenoid esters. The resins may be obtained from the wax as an extract. For example, JP 2003-95874A discloses mascara using a candelilla resin obtained by subjecting a candelilla wax to fractional extraction with an organic solvent. The concentration of free Candelilla resin in the present invention may be adjusted according to the needs of the specific formulation, but will generally be from 0.1 to 15% by weight of the composition, more preferably 1 to 10%, more preferably still 2% to 5% by weight of the final composition. By “free Candelilla resin”, we mean resin that is not trapped in the Candelilla wax. The resin is oil soluble, and therefore, may preferably be included in an oil phase of the mascara composition. Candelilla wax itself is not a required component of the invention, only the resin. Preferably, the wax is not used in compositions according to the invention, or if it is used, the amount of resin trapped in the wax is not part of the resin concentration mentioned above. Together, the Candelilla wax resin and pullulan make up an all natural film forming system.

### Rosin

A third component of the invention is one or more rosins (a.k.a. colophony, colophonium, colophonia) or rosin derivatives. By rosin, we mean a natural resin of a coniferous plant or tree, such as pine rosin. For example, a rosin having a number-average molecular weight of less than or equal to 10,000 may be useful. The rosin(s) and/or rosin derivatives may be present in the composition according to the invention, at concentrations ranging from 0.1% to 20% by weight, preferably from 0.5% to 15% by weight, and better still from 1% to 10% by weight, relative to the total weight of the composition. Rosin may advantageously be added to an oil phase of the composition.

Naturally derived rosin derivatives obtained by polymerization or hydrogenation may also be useful. For example, glycosyl rosinate and propanediol rosinates. A preferred rosin derivative is obtained by esterification of rosin acids with pentaerythritol. A particularly useful rosin derivative is dipentaerythrityl hexahydroxystearate/hexastearate/hexarosinate (tradename: Cosmol 168ARV™) sold by the company Nisshin OilliO Group, Ltd, Tokyo, JP. When dipentaerythrityl hexahydroxystearate/hexastearate/hexarosinate is used, mascara compositions according to the present invention may comprise about 0.5% to 6% of that material.

### Sebacic Acid/Hydrogenated Castor Oil Copolymer

A fourth component of the invention is a sebacic acid/hydrogenated castor oil copolymer. Such a naturally derived material is sold by SpecialChem, under the tradename Crodabond CSA™. In compositions of the present invention, this material may be used from 0.1 to 20%; preferably 1.0-10%; and more preferably 1.0% to 5%, by weight of the total composition. Sebacic acid/hydrogenated castor oil copolymer may advantageously be added to an oil phase of the composition.

Together, the rosin and sebacic acid/hydrogenated castor oil act as plasticizers for the pullulan and candelilla wax resin. When plasticizers are used in commercially available mascara compositions, it is common to use petroleum based products, such as certain esters, ethers and ethoxylated compounds. In contrast, in the present invention, we were able to balance the natural film former system (pullulan and candelilla wax resin) with a naturally derived plasticizer system. Furthermore, as will be seen below, compositions utilizing only the first three essential ingredients, were still not commercially acceptable. It was not until all four ingredients were brought together, in the proper balance, that the performance of the resulting mascara composition was on par with commercially available mascara compositions. Preferably, the ratio of pullulan and candelilla wax resin to plasticizers (rosin and sebacic acid/hydrogenated castor oil) is from 1:2 to 2:1; more preferably, from 1:1.5 to 1.5:1; even more preferably, from 1:1.2 to 1.2:1.

The four essential ingredients, used properly, constitute a naturally-derived, non-animal-derived, hair fixative system. Advantageously, this system, absent any synthetic film formers or synthetic hair fixatives, has sufficient film forming capabilities to allow the system be used in the preparation of commercially acceptable hair fixative compositions, especially mascaras. It was not at all obvious that this system, absent any synthetic film formers or synthetic hair fixatives, could be used to make a mascara that provides excellent thickening (i.e. volumizing) and lengthening, without flaking or smudging for an extended wear time. In fact, it is surprising that such a high performing mascara could be achieved with a naturally derived, non-animal-derived hair fixative system, especially given the seemingly ubiquitous use of synthetic film former systems, and the initial failures of systems based on pullulan alone. Advantageously, the hair fixative system revealed herein uses no heavy metals, no crosslinking ingredients, no catalysts and no complexes, which may be harmful to the user and the environment.

The form of a composition of the present invention is not generally limited, although emulsions are preferred. An emulsion composition may be a water-in-oil or oil-in-water emulsion. In the form of an emulsion, the composition may contain from about 10% to 60%, preferably from about 20% - 50%, more preferably from about 30-40% water, and from about 1% to 75%, preferably from about 5% to 60%, more preferably from about 10% to 50% of oil. A preferred composition is an oil-in-water composition having 35% to 50% water

To the extent that they do not interfere with the performance of the required components of the invention, additional natural film formers may be included in a composition according to the invention, but these may not take the place of any of the components identified as essential, or the mascara performance described herein may not be achieved. Also, if any optional film formers are used, the balance between the film former system and the plasticizer system may need to be readjusted to meet defined standards of performance, if they can be met at all. Therefore, while the present invention does not exclude the use of optional film formers, it is preferable to limit optional film formers to no more than 20% by weight of the composition, more preferably no more than 10%; even more preferably no more than 5% by weight of the composition.

Optional, natural film formers include: Abies Balsamia resin, Aloe barbedensis leaf polysaccharides, Amorphallus konjac root extract, Arabinoxylan, Astragalus Gummifer gum, Avena Sativa protein, Brassica Campestris/Aleurites Fordii oil copolymer, Calcium carrageenan, Calcium Chitosan, Cellulose gum, Cera Alba, Chitosan, Copaifera Officianalis (Balsam Copaiba) Resin, Corn Starch Modified, Crambe Abyssinica seed oil phytosterol esters, Cyclodextrin crosspolymer, Dehydroxanthan gum, Ethylcellulose, Euphorbia Cerifera (candelilla) wax, Galactoarabian, Glycosyl trehalose, Hydrogenated Brassica Campestris/Aleurites Fordii Oil Copolymer, Hydrogenated Dimer Dilinoleic Acid/Hexanediol Copolymer, Hydrogenated Olive Oil Caprylyl Esters, Hydrogenated Olive Oil Cetyl Esters, Hydrogenated Olive Oil Decyl Esters, Hydrogenated Olive Oil Hexyl Esters, Hydrogenated Olive Oil Lauryl Esters, Hydrogenated Olive Oil Myristyl Esters, Hydrogenated Starch Hydrolysate, Hydroxyethylcellulose, Hydroxyethyl Chitosan, Hydroxyethyl Ethylcellulose, Hydroxypropylcellulose, Hydroxypropyl Chitosan, Lactobacillus/Arundinaria Gigantea Ferment Filtrate, Lactobacillus/Rice Ferment Filtrate, Levan, Maltodextrin, Maltooligosyl Glucoside, Mannan, Melia Azadirachta Conditioned Media/Culture, Myrica Cerifera (Bayberry) Fruit Wax, Myrica Pubescens Fruit Wax, Myroxylon Balsamum (Balsam Tolu) Resin, Myroxylon Pereirae (Balsam Peru) Resin, Pistacia Lentiscus (Mastic) Gum, Polianthes Tuberosa Extract, Potassium Carrageenan, Pseudotsuga Menziesii (Balsam Oregon) Resin, Rhizobian Gum, Rubber Latex, Sodium Hydroxypropyl Oxidized Starch Succinate, Triticum Aestivum (Wheat) Peptide, Triticum Vulgare (Wheat) Protein, Welan Gum, Yeast Beta-Glucan, Yeast Polysaccharides, Zein. Preferred natural film-formers are non-animal-derived. Also, preferred natural film formers have not been modified by non-natural processes, which expend energy and create waste products.

To the extent that they do not interfere with the performance of the required components of the invention, any cosmetically acceptable ingredients may be included in a composition according to the invention. Preferred are natural, non-animal derived ingredients. These may include one or more of the following: aqueous phase structuring agents oils, oil phase structuring agents, surfactants, emulsifiers, humectants, botanical extracts, sunscreens, particulate materials, powders, pigments, coatings, fiber materials, preservatives, vitamins, antioxidants, gellants, perfume, viscosity controlling agents, and agents affecting substantivity, shine, curl, volume, length, hold, etc. When surfactants are present in the composition, surfactants which are able to act as a coupler for film formers are preferable.

In the examples that follow, compositions A through I comprise pullulan, a resin of Candelilla wax and a rosin, but no sebacic acid/castor oil copolymer. Compositions J, K and L have all four essential components in differing ratios. The compositions are oil in water emulsions. At he bottom of each table is a screening qualitative evaluation of each composition.

### EXAMPLES A-F

| CTFA name | A | B | C | D | E | F |
|---|---|---|---|---|---|---|
| purified water | 43.7 | 42.0 | 42.3 | 43.75 | 41.8 | 38.25 |
| iron oxides | 12.0 | 12.0 | 10.0 | 10.0 | 10.0 | 10.0 |
| carnauba wax | 4.5 | 4.5 | 5.5 | 4.7 | 4.5 | 4.6 |
| stearic acid | 4.5 | 4.65 | 4.5 | 4.5 | 4.5 | 5.0 |
| silica | 4.2 | 4.2 | 4.0 | 3.5 | 3.5 | 3.5 |
| mica | 4.0 | 4.0 | 3.0 | 3.0 | 3.0 | 1.0 |
| cellulose | 4.0 | 4.0 | 3.6 | 3.8 | 3.8 | 4.0 |
| hydrogenated lecithin | 3.0 | 3.0 | 3.2 | 3.2 | 3.2 | 3.2 |
| orange peel wax | 3.0 | 3.75 | 3.6 | 3.8 | 3.8 | 4.2 |
| arginine | 3.0 | 3.0 | 3.0 | 3.0 | 3.0 | 3.0 |
| disodium coco-glucoside | 2.0 | 2.0 | 2.5 | 2.5 | 2.5 | 2.5 |
| citrate | | | | | | |
| Euphorbia Cerifera | 2.0 | 1.75 | 2.5 | 2.0 | 2.0 | 2.1 |
| (candelilla) wax extract | | | | | | |
| pullulan | 1.8 | 1.9 | 2.0 | 2.0 | 2.0 | 4.0 |
| dipentaerythrityl | 2.0 | 2.75 | 3.0 | 3.6 | 3.5 | 4.1 |
| hexahydroxystearate/ | | | | | | |
| hexastearate/hexarosinate | | | | | | |
| hydrogenated castor oil/ | — | — | — | — | — | — |
| sebacic acid copolymer | | | | | | |
| kaolin | 1.0 | 1.0 | 0.5 | 0.1 | 0.1 | 0.1 |
| water/aqua/eau/yeast | 1.0 | 1.0 | 1.5 | 1.5 | 1.5 | 1.65 |
| extract\faex\extrait de levuer/ | | | | | | |
| phenoxyethanol | | | | | | |
| water\aqua\eau/yeast extract | | | | | | |
| glyceryl caprylate | 1.0 | 1.0 | 1.0 | 1.0 | 1.0 | 1.0 |
| phenoxyethanol/caprylyl | 0.9 | 0.9 | 0.9 | 0.9 | 0.9 | 0.9 |
| glycol | | | | | | |
| sorbitol | 0.5 | 0.75 | 0.4 | 0.5 | 0.5 | 0.5 |
| water/polyaminopropyl | 0.5 | 0.5 | 0.5 | 0.5 | 0.5 | 0.5 |
| biguanide | | | | | | |
| dimethicone | 0.4 | 0.4 | 0.3 | 0.2 | 0.2 | 0.2 |
| sodium gluconate | 0.2 | 0.2 | 0.2 | 0.1 | 0.1 | 0.1 |
| acacia Senegal gum | 0.2 | 0.15 | 0.6 | 1.0 | 3.0 | 4.0 |
| althea officinalis root extract/ | 0.1 | 0.1 | 0.1 | 0.1 | 0.1 | 0.1 |
| maltodextrin | | | | | | |
| honey (mel) | 0.1 | 0.1 | 0.1 | 0.1 | 0.1 | 0.1 |
| aloe barbedensis leaf | 0.1 | 0.1 | 0.1 | 0.1 | 0.1 | 0.1 |
| gluconolactone/sodium | 0.1 | 0.1 | 0.1 | 0.1 | 0.1 | 0.1 |
| benzoate/calcium | | | | | | |
| gluconate | | | | | | |
| dehydroxanthan gum | 0.1 | 0.1 | 0.6 | 0.3 | 0.05 | 0.05 |
| pelargonium gravolens wax | 0.05 | 0.05 | 0.05 | 0.05 | 0.05 | 0.05 |
| jasminum officinale (jasmine) | 0.05 | 0.05 | 0.05 | 0.05 | 0.05 | 0.05 |
| flower wax | | | | | | |
| hydroxyethyl-cellulose | | | 0.3 | 0.05 | 0.05 | 0.05 |
| polyglyceryl-6 polyricinoleate | | | | | 0.5 | |
| polyglyceryl-10 | | | | | | 1.0 |
| polyricinoleate | | | | | | |
| glyceryl stearate | | | | | | |
| acrylates copolymer | | | | | | |
| oleic/linoleic/linolenic | | | | | | |
| polyglycerides | | | | | | |
| Volume | low | | | low | low | low |
| Flaking | high | high | high | | | |
| Clumping | | high | | high | | |
| Smudging | | high | | | | |
| Spiking | | | | high | | |

### EXAMPLES G-L

| CTFA name | G | H | I | J | K | L |
|---|---|---|---|---|---|---|
| purified water | 46.2 | 43.2 | 46.15 | 44.15 | 43.75 | 47.5 |
| iron oxides | 10.0 | 10.0 | 10.0 | 10.0 | 10.0 | 8.0 |
| carnauba wax | 4.5 | 4.5 | 4.5 | 5.0 | 6.0 | 6.0 |
| stearic acid | 4.0 | 4.0 | 2.0 | 2.0 | 2.0 | 2.0 |
| silica | 1.5 | 1.5 | 2.5 | 2.5 | 3.0 | 3.0 |
| mica | | | | | | |
| cellulose | 3.0 | 3.0 | 3.0 | 3.0 | 3.0 | 3.0 |
| hydrogenated lecithin | 3.0 | 3.0 | 3.0 | 3.0 | 3.0 | 1.0 |
| orange peel wax | 3.0 | 3.0 | 3.0 | 2.0 | 2.0 | 2.0 |
| arginine | 3.0 | 3.0 | 1.0 | 1.0 | 1.0 | 1.0 |
| disodium coco-glucoside | 0.5 | 0.5 | 0.5 | 0.5 | 0.5 | |
| citrate | | | | | | |
| Euphorbia Cerifera | 2.0 | 2.0 | 2.5 | 2.0 | 2.5 | 3.0 |
| (candelilla) wax extract | | | | | | |
| pullulan | 2.0 | 2.0 | 2.0 | 2.0 | 3.0 | 3.0 |
| dipentaerythrityl | 5.5 | 5.5 | 3.0 | 3.0 | 2.0 | 1.0 |
| hexahydroxystearate/ | | | | | | |
| hexastearate/hexarosinate | | | | | | |
| hydrogenated castor oil/ | — | — | — | 5.0 | 4.0 | 4.0 |
| sebacic acid copolymer | | | | | | |
| kaolin | 0.1 | 0.1 | 0.1 | 0.1 | 0.1 | 0.1 |
| water/aqua/eau/yeast extract\ | 1.5 | 1.5 | 1.5 | 1.5 | 1.5 | 1.5 |
| faex\extrait de levuer/ | | | | | | |
| phenoxyethanol | | | | | | |
| water\aqua\eau/yeast extract | | | | | 1.0 | 1.0 |
| glyceryl caprylate | 0.5 | 0.5 | 0.5 | 0.5 | 0.5 | 1.0 |
| phenoxyethanol/caprylyl | 0.9 | 0.9 | 0.9 | 0.9 | 0.9 | 1.5 |
| glycol | | | | | | |
| sorbitol | 0.25 | 0.25 | 0.2 | 0.2 | 0.1 | 0.1 |
| water/polyaminopropyl | 0.5 | 0.5 | 0.5 | 0.5 | 0.5 | 0.5 |
| biguanide | | | | | | |
| dimethicone | 0.05 | 0.05 | 0.05 | 0.05 | 0.45 | 0.1 |
| sodium gluconate | 0.1 | 0.1 | 0.1 | 0.1 | 0.1 | 0.1 |
| acacia Senegal gum | 2.0 | 2.0 | 2.0 | 2.0 | 2.2 | 2.5 |
| althea officinalis root extract/ | 0.1 | 0.1 | 0.1 | 0.1 | 0.1 | 0.1 |
| maltodextrin | | | | | | |
| honey (mel) | 0.1 | 0.1 | 0.1 | 0.1 | 0.1 | 0.1 |
| aloe barbedensis leaf | 0.1 | 0.1 | 0.1 | 0.1 | 0.1 | 0.1 |
| gluconolactone/sodium | 0.1 | 0.1 | 0.1 | 0.1 | 0.1 | 0.1 |
| benzoate/calcium gluconate | | | | | | |
| dehydroxanthan gum | 0.1 | 0.1 | 0.1 | 0.1 | 0.1 | 0.1 |
| pelargonium gravolens wax | 0.05 | 0.05 | 0.05 | 0.05 | | |
| jasminum officinale (jasmine) | 0.05 | 0.05 | 0.05 | 0.05 | | |
| flower wax | | | | | | |
| hydroxyethylcellulose | 0.3 | 0.3 | 0.4 | 0.4 | 0.4 | 0.6 |
| polyglyceryl-6 polyricinoleate | | | | | | |
| polyglyceryl-10 | 2.0 | 2.0 | 4.0 | 4.0 | 2.0 | 2.0 |
| polyricinoleate | | | | | | |
| glyceryl stearate | 3.0 | 3.0 | 3.0 | 4.0 | 4.0 | 4.0 |
| acrylates copolymer | | 3.0 | | | | |
| oleic/linoleic/linolenic | | | 3.0 | | | |
| polyglycerides | | | | | | |
| Volume | low | low | low | low | low | high |
| Flaking | | | | high | high | low |
| Clumping | | | | high | high | low |
| Smudging | | | | high | high | low |
| Spiking | | | | | | low |

Following this screening of various formulae, the performance of Composition L was measured for various parameters.

Protocol

Adult women who were interested in taking part in this study were recruited. To be considered as a potential test subject, each candidate must have possessed medium eyelashes; expressed willingness to cooperate with the investigator; convinced the investigator that she was dependable and would comply with the study regimen; demonstrated the ability to understand the purpose of the study and what was required of her to bring it to a meaningful conclusion; demonstrated the ability to understand what risks were associated with participation; demonstrated the ability to read and understand all the items in the informed consent document; signed the informed consent document of her own free will and without any reservations. A prospective participant was excluded for any of the following: a systemic illness that contra-indicated participation; any dermatological disorders in the areas that were to be used in the study; may not be pregnant women or lactating mothers; using systemic or topical retinoids, antihistamines or similar agents.

Test Subjects: Ten women who satisfied all the requirements itemized in the list of inclusion and exclusion criteria.

Method of Application: The test subjects were instructed to wear no makeup or moisturizer on the day of testing. The test subjects applied forty strokes to the upper lashes. Then they re-charged the mascara and applied forty strokes to the lower lashes. The test subjects were not allowed to pump the mascara.

Methods of evaluation included analysis of images and observations by trained experts. Evaluations were made immediately after application of the product to the eyelashes, and after 8 hours of wear. Evaluations immediately after application served as a baseline. The following results were statistically significant at a confidence level of p<0.05.

Image Analysis—the Performance of Composition L

| | Immediate | 8 hours |
|---|---|---|
| Length | 34% increase | 25% increase |
| Volume | 112% increase | 98% increase |
| Curl | 15° increase | 12° increase |

Trained Expert Panel—the Performance of Composition L

| Parameter | Immediate | 8 hours |
|---|---|---|
| Separation | 20% increase | 17% increase |
| Thickness | 33% increase | 31% increase |
| Spiking | 23% increase | 23% increase |
| Clumping | 12% increase | 12% increase |
| Flaking | — | 10% increase |
| Smudging | — | 10% increase |
| Wear | — | 12% loss of product |
| Overall Look | 29% improvement | 27% improvement |
| Ease of removal | — | 7.3/10 |

As can be seen, the performance of composition L is very good, especially as to volumizing, lengthening and thickening. Composition L, with a naturally derived hair fixative system according to the present invention, was further tested by comparing the performance of compositions A and L, and two commercially marketed compositions, Estee Lauder More Than Mascara and Clinique High Impact Mascara. The method of evaluation was observations by trained experts. Some parameters are evaluated immediately after application of the product to the eyelashes, and some are measured after 8 hours of wear. Evaluations immediately after application served as a baseline. The following results were statistically significant at a confidence level of p<0.05. For curl, separation, length, volume, thickness and overall look, a higher percentage indicates better performance. For spiking, clumping, flaking, smudging and wear a lower score indicates better performance. For wear, a score of less than 20% indicates long wearability.

| | | | | | Estee Lauder | | Clinique | |
|---|---|---|---|---|---|---|---|---|
| | | | | | More Than | | High Impact | |
| | Composition A | | Composition L | | Mascara | | Mascara | |
| | | 8 | | 8 | | 8 | | 8 |
| | Immediate | hours | Immediate | hours | Immediate | hours | Immediate | hours |
| Curl | 29% | | 31% | 28% | 29% | | 36% | |
| Separation | 22% | | 19% | 17% | 23% | | 26% | |
| Length | 31% | | 34% | 33% | 32% | | 35% | |
| Volume | 20% | | 24% | 22% | 15% | | 22% | |
| Thickness | 24% | | 34% | 33% | 22% | | 28% | |
| Spiking | 22% | | 22% | 22% | 29% | | 25% | |
| Clumping | 12% | | 13% | 11% | 13% | | 15% | |
| Overall | 27% | | 30% | 26% | 28% | | 34% | |
| Look | | | | | | | | |
| Flaking | | 9% | — | 11% | | 8% | | 7% |
| Smudging | | 12% | — | 11% | | 11% | | 11% |
| Wear | | 15% | — | 13% | | 12% | | 11% |

Composition L, having a naturally derived hair fixative system as described herein, is clearly shown to be as effective or better in lengthening, thickening, volumizing and overall look, as the commercially available mascaras. Composition L is clearly shown to be significantly better than Composition A in these same categories. In contrast, composition A performed less well than both of the commercially available mascaras for lengthening, and not as well as one of them for volumizing, thickening and overall look.

What we claim is:

1. A mascara composition comprising a naturally derived hair fixative system, the hair fixative system comprising:
a natural, polymeric film forming system that comprises pullulan and candelilla wax resin; and
a naturally derived plasticizer system that comprises one or more rosins and/or rosin derivatives, and a sebacic acid/castor oil copolymer.

2. The mascara composition of claim 1 where in the ratio of natural film forming system to naturally derived plasticizer system is 1:2 to 2:1.

3. The mascara composition of claim 2 wherein the pullulan has a polymerization degree of 10-10,000.

4. The mascara composition of claim 3 wherein the pullulan is myristoyl pullulan.

5. The mascara composition of claim 2 wherein the concentration of pullulan is from 0.1% to 15% by weight of the composition.

6. The mascara composition of claim 2 wherein the concentration of free Candelilla resin is from 0.1 to 15% by weight of the composition.

7. The mascara composition of claim 6 having no candelilla wax.

8. The mascara composition of claim 2 wherein the concentration of rosins and/or rosin derivatives is from 0.1 to 20% by weight of the composition.

9. The mascara composition of claim 8 wherein at least one rosin has a number-average molecular weight of less than or equal to 10,000.

10. The mascara composition of claim 8 comprising one or more rosin derivatives selected from a glycosyl rosinate, a propanediol rosinate, and dipentaerythrityl hexahydroxystearate/hexastearate/hexarosinate.

11. The mascara composition of claim 8 comprising 0.5% to 6% of dipentaerythrityl hexahydroxystearate/hexastearate/hexarosinate.

12. The mascara composition of claim 2 wherein the concentration of sebacic acid/hydrogenated castor oil copolymer is from 0.1 to 20% by weight of the composition.

13. The mascara composition of claim 2 wherein the composition is an emulsion comprising from about 10% to 60% water and about 1% to 75% of oil.

14. The mascara composition of claim 13 that is an oil-in-water composition having 35% to 50% water.

15. The mascara composition of claim 14 wherein the candelilla wax resin, the rosins and/or rosin derivatives, and the sebacic acid/hydrogenated castor oil copolymer are included in an oil phase of the composition.

16. The mascara composition of claim 2 further comprising additional film formers other than the pullulan and candelilla wax resin system, the concentration of the additional film formers being no more than 20% by weight of the composition.

17. A naturally derived hair fixative system comprising:
a natural, polymeric film forming system that comprises pullulan and candelilla wax resin; and
a naturally derived plasticizer system that comprises one or more rosins and/or rosin derivatives, and a sebacic acid/castor oil copolymer.
